(19) United States
(12) Patent Application Publication
(10) Pub. No.: US 2009/0092662 A1
(43) Pub. Date: Apr. 9, 2009

(54) LIPOSOME FORMULATIONS OF BORONIC ACID COMPOUNDS

(76) Inventors: Anthony Huang, Saratoga, CA (US); Bing Luo, Fremont, CA (US); Jingkang Wang, San Francisco, CA (US); Yuanpeng Zhang, Cupertino, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/195,795

(22) Filed: Aug. 21, 2008

Related U.S. Application Data
(60) Provisional application No. 60/957,049, filed on Aug. 21, 2007.

(51) Int. Cl. A61K 9/127; A61K 31/69; A61P 35/00

(52) U.S. Cl. 424/450; 514/64

(57) ABSTRACT

A liposome composition comprised of liposomes having a peptide boronic acid proteasome inhibitor compound entrapped in the liposomes is described. More specifically, liposomes having a compound of Formula I or II entrapped in the interior aqueous compartment are loaded with a peptide boronic acid compound, to form a boronate ester compound inside the liposomal aqueous compartment. In one embodiment, the liposomes have an outer coating of hydrophilic polymer chains and are used to treat a solid tumor in a subject.

### CROSS REFERENCE TO RELATED APPLICATIONS

The present application claim priority to U.S. Provisional Patent Application Ser. No. 60/957,049, filed on Aug. 21, 2007, which is hereby incorporated by reference.

### TECHNICAL FIELD

A liposome composition comprising a boronic acid compound, and in particular a peptide boronic acid compound, is provided. More specifically, a liposome composition comprising a boronic acid compound and a compound of Formula I and/or II as defined below, is provided.

### BACKGROUND

Liposomes, or lipid bilayer vesicles, are spherical vesicles comprised of concentrically ordered lipid bilayers that encapsulate an aqueous phase. Liposomes serve as a delivery vehicle for therapeutic and diagnostic agents contained in the aqueous phase or in the lipid bilayers. Delivery of drugs in liposome-entrapped form can provide a variety of advantages, depending on the drug, including, for example, a decreased drug toxicity, altered pharmacokinetics, or improved drug solubility. Liposomes when formulated to include a surface coating of hydrophilic polymer chains, i.e., so-called STEALTH® or long-circulating liposomes, offer the further advantage of a long blood circulation lifetime, due in part to reduced removal of the liposomes by the mononuclear phagocyte system. Often an extended lifetime is necessary in order for the liposomes to reach their desired target region or cell from the site of injection.

Ideally, such liposomes can be prepared to include an entrapped therapeutic or diagnostic compound (i) with high loading efficiency, (ii) at a high concentration of entrapped compound, and (iii) in a stable form, i.e., with little compound leakage on storage. One particularly interesting class of therapeutic compounds are peptide boronic acid compounds which are peptide proteasome inhibitor compounds containing an α-aminoboronic acid at the acidic, or C-terminal, end of the peptide sequence. One such peptide boronic acid compound is bortezomib, previously known as PS-341 (VELCADE®, Millennium Pharmaceuticals, Inc, Cambridge, Mass.). Bortezomib is a dipeptide boronic acid derivative and was synthesized as a highly selective, potent, reversible proteasome inhibitor with a Kᵢ of 0.6 nmol/L (Adams, Semin. Oncol. 28(6):613-619 (2001)). Using the National Cancer Institute's in vitro screen, bortezomib showed cytotoxicity against a range of tumor lines (Adams, Id.) and had antitumor activity in human prostate (Frankel et al., Clin. Cancer Res. 6(9):3719-3728 (2000); DiPaola et al., Hematol. Oncol. Clin. North Am. 15(3):509-524 (2001)) and lung cancer xenograft models (Oyaizu et al., Oncol. Rep. 8(4):825-829 (2001)).

Liposome compositions comprised of liposomes having a peptide boronic proteasome inhibitor compound entrapped in the liposome are described in the International Application No. WO 2006/052734 published under the Patent Cooperation Treaty on May 18, 2006. The boronic acid compound is entrapped in the liposome in the form of a boronate ester, subsequent to interaction with a liposome-entrapped polyol. In one embodiment, the liposome entrapped polyol is a monomeric or polymeric compound containing alcoholic hydroxyl groups, wherein the polyol can be an aliphatic compound, a ring compound diol, a polyphenol, or the like. In another embodiment, the monomeric polyol includes sugars, glycerol, glycols, carbohydrates, amino-sugars (especially amino-sorbitol), sugar-alcohols, deoxysorbitol, gluconic acid, tartaric acid, gallic acid, etc.

It would be desirable to entrap such peptide boronic acid compounds into a liposomal carrier. However, there are difficulties associated with how to efficiently load these relatively non-polar dipeptides and retain them within the liposome in a stable entrapped manner. More particularly, the subject matter relates to liposomes prepared from components that improve loading and retention of a peptide boronic acid compound within the liposomes.

### SUMMARY

Accordingly, it is an object to provide a liposome composition comprising a peptide boronic acid compound stably entrapped in the liposomes.

It is another object to provide a suspension of liposomes having a peptide boronic acid compound entrapped in the liposomes in the stable form of a peptide boronate ester.

In one embodiment, a liposome composition is provided, comprising a peptide boronic acid compound that is stably entrapped in the liposomes wherein the peptide boronic acid compound is a dipeptidyl boronic acid compound. An exemplary dipeptidyl boronic acid compound is bortezomib.

In one aspect, a composition is provided, comprising liposomes formed of a vesicle-forming lipid, and entrapped in said liposomes, a boronate ester compound comprised of a peptide boronic acid compound and a compound of Formula I.

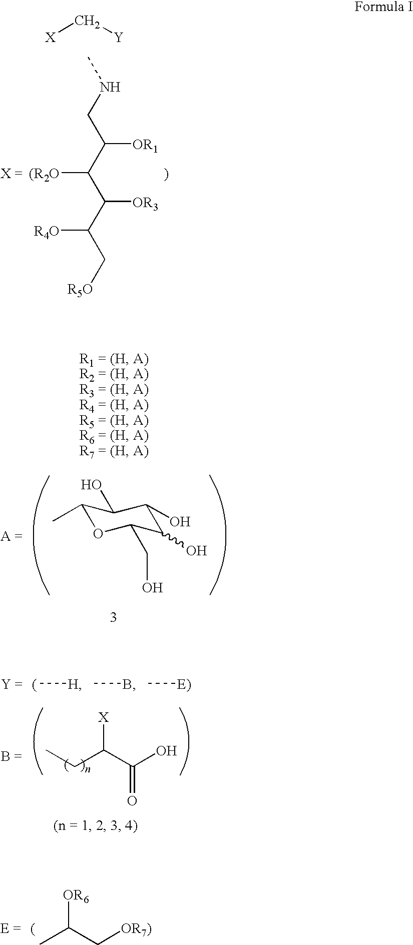

wherein X, Y, A, B, E, R₁, R₂, R₃, R₄, R₅, R₆ and R₇ are as described above, provided, however, that Y, R₁, R₂, R₃, R₄ and R₅ are not each H, and any enantiomers or diastereoisomers.

In another embodiment, the compound of Formula I is one wherein Y is H. An exemplary compound is where Y is H and R₁ is H. Another exemplary compound is where Y is H and R₂ is H. Another exemplary compound is where Y is H and R₃ is H. Another exemplary compound is where Y is H and R₄ is H. Another exemplary compound is where Y is H and R₅ is H. Another exemplary compound is where Y is H and R₃ is A. In particular, the compound is where Y, R₁, R₂, R₄, R₅ are H and R₃ is A.

In another embodiment, the compound of Formula I is one wherein Y is B. An exemplary compound is where Y is B and R₁ is H. Another exemplary compound is where Y is B and R₂ is H. Another exemplary compound is where Y is B and R₃ is H. Another exemplary compound is where Y is B and R₄ is H. Another exemplary compound is where Y is B and R₅ is H. In particular, the compound is where Y is B and R₁, R₂, R₃, R₄ and R₅ are H. Another exemplary compound is where Y is B and R₃ is A. In particular, the compound is where Y is B and R₁, R₂, R₄, R₅ are H and R₃ is A.

In another embodiment, the compound of Formula I is one wherein Y is E. An exemplary compound is where Y is E and R₁ is H. Another exemplary compound is where Y is E and R₂ is H. Another exemplary compound is where Y is E and R₃ is H. Another exemplary compound is where Y is E and R₄ is H. Another exemplary compound is where Y is E and R₅ is H. Another exemplary compound is where Y is E and R₆ is H. Another exemplary compound is where Y is E and R₇ is H. In particular, the compound is where Y is E and R₁, R₂, R₃, R₄, R₅, R₆ and R₇ are H. Another exemplary compound is where Y is E and R₃ is A. In particular, the compound is where Y is E and R₁, R₂, R₄, R₅, R₆, R₇ are H and R₃ is A.

In one aspect, a composition is provided, comprising liposomes formed of a vesicle-forming lipid, and entrapped in said liposomes, a boronate ester compound comprised of a peptide boronic acid compound and a compound of Formula II which is a conjugated dendrimer.

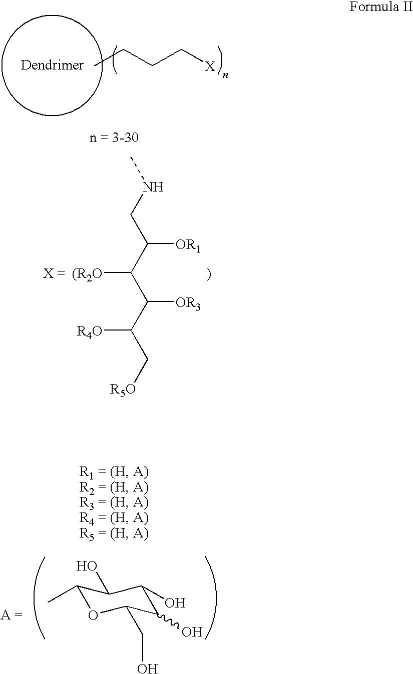

wherein D, X, A, R₁, R₂, R₃, R₄ and R₅ are as described above.

In one embodiment, the compound of Formula II is one wherein the dendrimer is a G₁ or G₂ dendrimer. An exemplary compound is where the dendrimer is G₁. Another exemplary compound is where the dendrimer is G₂.

In another embodiment, the compound of Formula II is one wherein n is an integer between 3 and 30. An exemplary compound is where R₁ is H. Another exemplary compound is where R₂ is H. Another exemplary compound is where R₃ is H. Another exemplary compound is where R₄ is H. Another exemplary compound is where R₅ is H. In particular, the compound is where R₁, R₂, R₃, R₄ and R₅ are H. In particular, the compound is a conjugated G₂ dendrimer. In one embodiment, the G₂ dendrimer has 16 amino groups per molecule.

In another embodiment, the liposomes further comprise a higher inside/lower outside ion gradient. The ion gradient can be, for example, a hydrogen ion (pH) gradient. When the ion gradient is a pH gradient, the inside pH of the liposomes can be between about 7.5-8.5 and the pH of the environment outside the liposomes can be between about 6-7.

In another embodiment, the liposomes further include between about 1-20 mole percent of a hydrophobic moiety derivatized with a hydrophilic polymer.

In embodiments where the liposomes includes a hydrophobic moiety covalently linked to a hydrophilic polymer, a preferred polymer is polyethylene glycol. A preferred hydrophobic moiety is a lipid, and is preferably a vesicle-forming lipid.

In another aspect, a method of delivering a peptide boronic acid compound, comprising preparing a suspension of liposomes in an aqueous solution, the liposomes having an entrapped peptide boronic acid compound covalently attached to a compound of Formula I or II to form a peptidyl boronate ester compound, and administering the suspension of liposomes to a subject is provided.

In one embodiment, the liposomes are administered by injection.

In another aspect, a method of selectively destroying tumor tissue in a tumor-bearing subject undergoing radiation therapy, comprising administering to a tumor-bearing subject, liposomes having an entrapped peptide boronic acid covalently attached to a compound of Formula I or II to form a peptidyl boronate ester compound and an isotope of boron; and subjecting said subject to neutron-radiation therapy is provided.

In one embodiment, the isotope of boron is in the peptide boronic acid, such as ¹⁰B.

In addition to the exemplary aspects and embodiments described above, further aspects and embodiments will become apparent by reference to the drawings and by study of the following descriptions.

### BRIEF DESCRIPTION OF THE DRAWINGS

FIGS. 1A-1C show the structures of exemplary peptide boronic acid compounds; and

FIG. 2 illustrates loading of an exemplary peptide boronic acid into a liposome against a higher inside/lower outside pH gradient for formation of a boronate ester inside the liposome.

### DETAILED DESCRIPTION

### I. Definitions

“Peptide boronic acid compound” intends a compound of the form

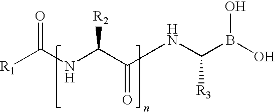

where R¹, R², and R³ are independently selected moieties that can be the same or different from each other, and n is from 1-8, preferably 1-4.

A “hydrophilic polymer” intends a polymer having some amount of solubility in water at room temperature. Exemplary hydrophilic polymers include polyvinylpyrrolidone, polyvinylmethylether, polymethyloxazoline, polyethyloxazoline, polyhydroxypropyloxazoline, polyhydroxypropylmethacrylamide, polymethacrylamide, polydimethylacrylamide, polyhydroxypropylmethacrylate, polyhydroxyethylacrylate, hydroxymethylcellulose, hydroxyethylcellulose, polyethyleneglycol, polyaspartamide and hydrophilic peptide sequences. The polymers may be employed as homopolymers or as block or random copolymers. A preferred hydrophilic polymer chain is polyethyleneglycol (PEG), preferably as a PEG chain having a molecular weight between 500-10,000 daltons, more preferably between 750-10,000 daltons, still more preferably between 750-5,000 daltons.

“Higher inside/lower outside pH gradient” refers to a transmembrane pH gradient between the interior of liposomes (higher pH) and the external medium (lower pH) in which the liposomes are suspended. Typically, the interior liposome pH is at least 1 pH unit greater than the external medium pH, and preferably 2-4 units greater.

“Liposome entrapped” intends refers to a compound being sequestered in the central aqueous compartment of liposomes, in the aqueous space between liposome lipid bilayers, or within the bilayer itself.

### II. Liposome Formulation

In one aspect, the invention provides a liposome composition having an entrapped peptide boronic acid compound. In this section, the liposome composition and method of preparation will be described.

A. Liposome Components

As noted above, the liposome formulation is comprised of liposomes containing an entrapped peptide boronic acid compound. Peptide boronic acid compounds are peptides containing an α-aminoboronic acid at the acidic, or C-terminal, end of the peptide sequence. In general, peptide boronic acid compounds are of the form:

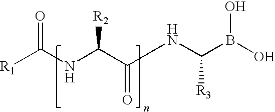

where R₁, R₂, and R₃ are independently selected moieties that can be the same or different from each other, and n is from 1-8, preferably 1-4. Compounds having an aspartic acid or glutamic acid residue with a boronic acid as a side chain are also contemplated.

Preferably, R₁, R₂, and R₃ are independently selected from hydrogen, alkyl, alkoxy, aryl, aryloxy, aralkyl, aralkoxy, cycloalkyl, or heterocycle; or any of R₁, R₂, and R₃ may form a heterocyclic ring with an adjacent nitrogen atom in the peptide backbone. Alkyl, including the alkyl component of alkoxy, aralkyl and aralkoxy, is preferably 1 to 10 carbon atoms, more preferably 1 to 6 carbon atoms, and may be linear or branched. Aryl, including the aryl component of aryloxy, aralkyl, and aralkoxy, is preferably mononuclear or binuclear (i.e. two fused rings), more preferably mononuclear, such as benzyl, benzyloxy, or phenyl. Aryl also includes heteroaryl, i.e. an aromatic ring having one or more nitrogen, oxygen, or sulfur atoms in the ring, such as furyl, pyrrole, pyridine, pyrazine, or indole. Cycloalkyl is preferably 3 to 6 carbon atoms. Heterocycle refers to a non-aromatic ring having one or more nitrogen, oxygen, or sulfur atoms in the ring, preferably a 5- to 7-membered ring having 3 to 6 carbon atoms. Such heterocycles include, for example, pyrrolidine, piperidine, piperazine, and morpholine. Either of cycloalkyl or heterocycle may be combined with alkyl; e.g. cyclohexylmethyl.

Any of the above groups (excluding hydrogen) may be substituted with one or more substituents selected from halogen, preferably fluoro or chloro; hydroxy; lower alkyl; lower alkoxy, such as methoxy or ethoxy; keto; aldehyde; carboxylic acid, ester, amide, carbonate, or carbamate; sulfonic acid or ester; cyano; primary, secondary, or tertiary amino; nitro; amidino; and thio or alkylthio. Preferably, the group includes at most two such substituents.

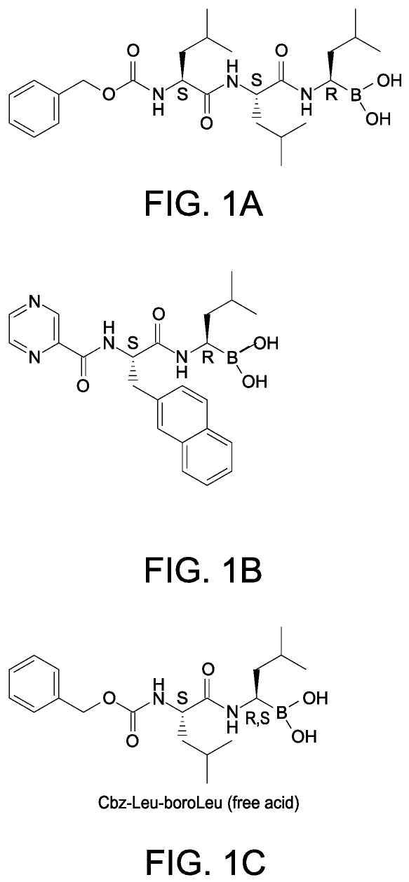

Exemplary peptide boronic acid compounds are shown in FIGS. 1A-1C. Specific examples of R₁, R₂, and R₃ shown in FIGS. 1A-1C include n-butyl, isobutyl, and neopentyl (alkyl); phenyl or pyrazyl (aryl); 4-((t-butoxycarbonyl)amino)butyl, 3-(nitroamidino)propyl, and (1-cyclopentyl-9-cyano)nonyl (substituted alkyl); naphthylmethyl and benzyl (aralkyl); benzyloxy (aralkoxy); and pyrrolidine (R₂ forms a heterocyclic ring with an adjacent nitrogen atom).

In general, the peptide boronic acid compound can be a mono-peptide, di-peptide, tri-peptide, or a higher order peptide compound. Other exemplary peptide boronic acid compounds are described in U.S. Pat. Nos. 6,083,903, 6,297,217, and 6,617,317, which are incorporated by reference herein.

Peptide boronic acids such as bortezomib are derivatives of usually short 2-4 amino acid peptides containing aminoboronic acid at the acidic end, C-terminal end, of the sequence (Zembower et al., Int. J. Pept. Protein Res. 47(5):405-413 (1996)). Due to the ability to form a stable tetrahedral borate complex between the boronic acid group and the active site serine or histidine moiety, peptide boronic acids are powerful serine-protease inhibitors. This activity is often enhanced and made highly specific towards a particular protease by varying the sequence of the peptide boronic acids and introducing unnatural amino acid residues and other substituents. This led to the selection of peptide boronic acids with powerful antiviral (Priestley, E. S. and Decicco, C. P., Org. Lett. 2(20):3095-3097 (2000); Bukhtiyarova, M. et al., Antivir. Chem. Chemother. 12(6):367-73 (2001); Archer, S. J. et al., Chem. Biol. 9(1):79-92 (2002); Priestley, E. S. et al., Bioorg. Med. Chem. Lett. 12(21):3199-202 (2002)) and cytotoxic activities (Teicher, B. A. et al., Clin. Cancer Res., 5(9):2638-45 (1999); Frankel et al., Clin. Cancer Res. 6(9):3719-28 (2000); Lightcap, E. S. et al., Clin. Chem. 46(5):673-83 (2000); Adams, J., Semin. Oncol., 28(6):613-19 (2001); Cusack, J. C., Jr. et al., Cancer Res. 61(9):3535-40 (2001); Shah, S. A. et al., J. Cell. Biochem. 82(1):110-22, (2001); Adams, J., Curr. Opin. Chem. Biol. 6(4):493-500 (2002); Orlowski, R. Z. and Dees, E. C., Breast Cancer Res. 5(1):1-7 (2002); Orlowski, R. Z. et al., J. Clin. Oncol. 20(22):4420-27, (2002); Schenkein, D., Clin. Lymphoma 3(1):49-55 (2002); Ling, Y. H. et al., Clin Cancer Res 9(3):1145-54 (2003)). These derivatives suffer from the same problems as other short peptides, most notably very fast clearance and inability to reach the in vivo target site.

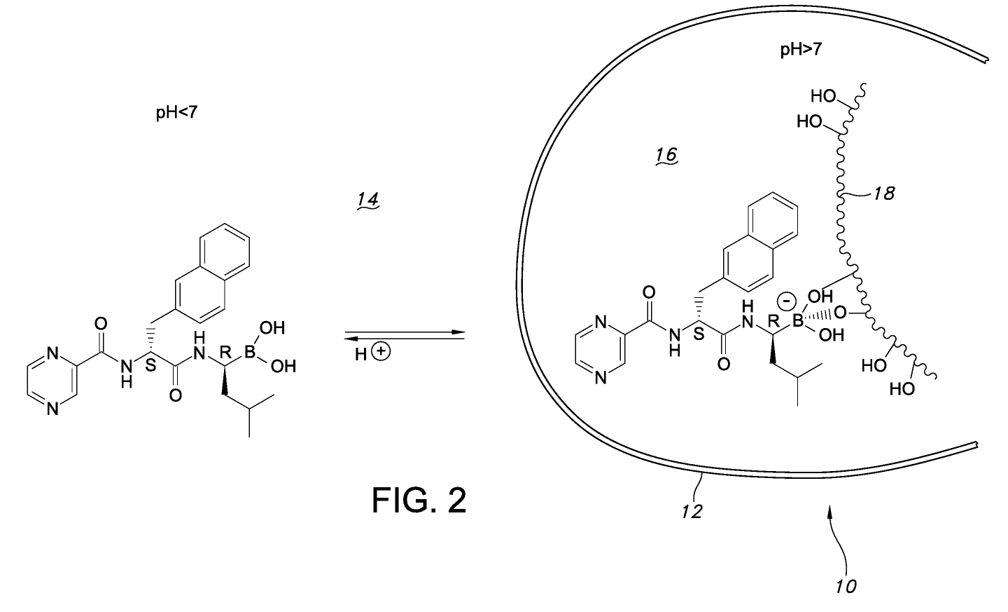

Many peptide boronic acid compounds lack an easily ionizable amino group, or are very polar, and thus are difficult to load into a liposome using conventional remote loading procedures discussed above. Thus, a loading method designed for peptide boronic acid compounds has been designed, to provide a liposome formulation where the peptide boronic acid compound is entrapped in the liposome in the form of a peptide boronate ester, as will now be described with respect to FIG. 2. FIG. 2 shows a liposome 10 having a lipid bilayer membrane represented by a single solid line 12. It will be appreciated that in multilamellar liposomes the lipid bilayer membrane is comprised of multiple lipid bilayers with intervening aqueous spaces. Liposome 10 is suspended in an external medium 14, where the pH of the external medium is lower than about 7.0, generally between about 5.5-7.0, more generally between 6.0-7.0. Liposome 10 has an internal aqueous compartment 16 defined by the lipid bilayer membrane. Entrapped within the internal aqueous compartment is a compound 18, preferably of a compound of Formula I. The compound of Formula I is preferably a moiety having multiple hydroxyl functionalities, and exemplary compounds are provided below. The pH of the internal aqueous compartment is preferably greater than about 7.0, more preferably between 7.1-9.0, still more preferably between 7.5 and 8.5.

Also entrapped in the liposome is a peptide boronic acid compound, represented in FIG. 2 by bortezomib. Bortezomib is also shown in the external aqueous medium, prior to passage across the lipid bilayer membrane. In the external aqueous medium, the compound is uncharged, due to the slightly acidic medium. In its uncharged state, the compound is freely permeable across the lipid bilayer. Formation of a boronate ester shifts the equilibrium to cause additional compound to permeate from the external medium across the lipid bilayer, leading to accumulation of the compound in the liposome. In another embodiment, the lower pH in the external suspension medium and the somewhat higher pH on the liposomal interior, combined with the complexing agent inside the liposome, induce drug accumulation into the liposome's aqueous internal compartment. Once inside the liposome, the compound reacts with the complexing agent to form a boronate ester. The boronate ester is essentially unable to cross the liposome bilayer, so that the drug compound, in the form of a boronate ester, accumulates inside the liposome.

The concentration of the complexing agent inside the liposomes is preferably such that the concentration of charged groups, e.g., hydroxyl groups, is greater than the concentration of boronic acid compound. In a composition having a final drug concentration of 100 mM, for example, the internal compound concentration of the polymer charge groups will typically be at least this great.

The complexing agent is present at a high-internal/low-external concentration; that is, there is a concentration gradient of the complexing agent across the liposome lipid bilayer membrane. If the complexing agent is present in significant amounts in the external bulk phase, the complexing agent reacts with the peptide boronic acid compound in the external medium, slowing accumulation of the compound inside the liposome. Thus, preferably, the liposomes are prepared, as described below, so that the composition is substantially free of the complexing agent in the bulk phase (outside aqueous phase).

Among various molecules that are suitable as complexing agents are compounds of Formula I or II set forth above. The compounds of Formula I or II form boronate esters. It is contemplated that the reactivity differences among the compounds of Formula I or II can be used to prepare liposome formulations with a gradient of entrapment strengths, thus fine-tuning the drug release characteristics. In general, the retention strength of the complexing reagents is related to the molecular weight and hydrophobic properties of the complexing reagents. The higher molecular weight and lower hydrophobic property of the complexing reagents gives longer retention of the peptide boronic acid compound(s).

In one embodiment, the compound of Formula I is one wherein Y is H. An exemplary compound is where Y is H and R₁ is H. Another exemplary compound is where Y is H and R₂ is H. Another exemplary compound is where Y is H and R₃ is H. Another exemplary compound is where Y is H and R₄ is H. Another exemplary compound is where Y is H and R₅ is H. Another exemplary compound is where Y is H and R₃ is A. In particular, the compound is where Y, R₁, R₂, R₄, R₅ are H and R₃ is A.

Compounds of Formula I wherein Y is H are commercially available or prepared according to the procedure set forth in Example 1.

In another embodiment, the compound of Formula I is one wherein Y is B. An exemplary compound is where Y is B and R₁ is H. Another exemplary compound is where Y is B and R₂ is H. Another exemplary compound is where Y is B and R₃ is H. Another exemplary compound is where Y is B and R₄ is H. Another exemplary compound is where Y is B and R₅ is H. In particular, the compound is where Y is B and R₁, R₂, R₃, R₄ and R₅ are H. Another exemplary compound is where Y is B and R₃ is A. In particular, the compound is where Y is B and R₁, R₂, R₄, R₅ are H and R₃ is A.

Compounds of Formula I wherein Y is B are commercially available or prepared according to the procedure set forth in Examples 2-3.

In another embodiment, the compound of Formula I is one wherein Y is E. An exemplary compound is where Y is E and R₁ is H. Another exemplary compound is where Y is E and R₂ is H. Another exemplary compound is where Y is E and R₃ is H. Another exemplary compound is where Y is E and R₄ is H. Another exemplary compound is where Y is E and R₅ is H. In particular, the compound is where Y is E and R₁, R₂, R₃, R₄ and R₅ are H. Another exemplary compound is where Y is E and R₃ is A. In particular, the compound is where Y is E and R₁, R₂, R₄, R₅ are H and R₃ is A.

Compounds of Formula I wherein Y is E are commercially available or prepared according to the procedure set forth in Example 5.

In one embodiment, the compound of Formula II is one wherein the dendrimer is a G₁ or G₂ dendrimer. An exemplary compound is where the dendrimer is G₁. Another exemplary compound is where the dendrimer is G₂.

In another embodiment, the compound of Formula II is a conjugated dendrimer. An exemplary compound is a compound of D wherein n is an integer from about 3 to about 30. An exemplary compound is where R₁ is H. Another exemplary compound is where R₂ is H. Another exemplary compound is where R₃ is H. Another exemplary compound is where R₄ is H. Another exemplary compound is where R₅ is H. In particular, the compound is where R₁, R₂, R₃, R₄ and R₅ are H. In particular, the compound is a conjugated G₂ dendrimer. In one embodiment, the G₂ dendrimer has 16 amino groups per molecule.

Compounds of Formula II are commercially available or prepared according to the procedure set forth in Example 4.

The liposomes in the composition are composed primarily of vesicle-forming lipids. Such a vesicle-forming lipid is one which can form spontaneously into bilayer vesicles in water, as exemplified by the phospholipids, with its hydrophobic moiety in contact with the interior, hydrophobic region of the bilayer membrane, and its head group moiety oriented toward the exterior, polar surface of the membrane. Lipids capable of stable incorporation into lipid bilayers, such as cholesterol and its various analogs, can also be used in the liposomes. The vesicle-forming lipids are preferably lipids having two hydrocarbon chains, typically acyl chains, and a head group, either polar or nonpolar. There are a variety of synthetic vesicle-forming lipids and naturally-occurring vesicle-forming lipids, including the phospholipids, such as phosphatidylcholine, phosphatidylethanolamine, phosphatidic acid, phosphatidylinositol, and sphingomyelin, where the two hydrocarbon chains are typically between about 14-22 carbon atoms in length, and have varying degrees of unsaturation. The above-described lipids and phospholipids whose acyl chains have varying degrees of saturation can be obtained commercially or prepared according to published methods. Other suitable lipids include glycolipids, cerebrosides and sterols, such as cholesterol.

The vesicle-forming lipid can be selected to achieve a specified degree of fluidity or rigidity, to control the stability of the liposome in serum, and/or to control the rate of release of the entrapped agent in the liposome. Liposomes having a more rigid lipid bilayer, or a liquid crystalline bilayer, are achieved by incorporation of a relatively rigid lipid, e.g., a lipid having a relatively high phase transition temperature, e.g., up to 60° C. Rigid, i.e., saturated, lipids contribute to greater membrane rigidity in the lipid bilayer. Other lipid components, such as cholesterol, are also known to contribute to membrane rigidity in lipid bilayer structures. On the other hand, lipid fluidity is achieved by incorporation of a relatively fluid lipid, typically one having a lipid phase with a relatively low liquid to liquid-crystalline phase transition temperature, e.g., at or below room temperature.

The liposomes can optionally include a vesicle-forming lipid covalently linked to a hydrophilic polymer. As has been described, for example in U.S. Pat. No. 5,013,556, including such a polymer-derivatized lipid in the liposome composition forms a surface coating of hydrophilic polymer chains around the liposome. The surface coating of hydrophilic polymer chains is effective to increase the in vivo blood circulation lifetime of the liposomes when compared to liposomes lacking such a coating. Polymer-derivatized lipids comprised of methoxy(polyethylene glycol) (mPEG) and a phosphatidylethanolamine (e.g., dimyristoyl phosphatidylethanolamine, dipalmitoyl phosphatidylethanolamine, distearoyl phosphatidylethanolamine (DSPE), or dioleoyl phosphatidylethanolamine) can be obtained from Avanti Polar Lipids, Inc. (Alabaster, Ala.) at various mPEG molecular weights (350, 550, 750, 1,000, 2,000, 3,000, and 5,000 Daltons). Lipopolymers of mPEG-ceramide can also be purchased from Avanti Polar Lipids, Inc. Preparation of lipid-polymer conjugates is also described in the literature, see U.S. Pat. Nos. 5,631,018, 6,586,001, and 5,013,556; Zalipsky, S. et al., Bioconjugate Chem. 8:111 (1997); Zalipsky, S. et al., Meth. Enzymol. 387:50 (2004). These lipopolymers can be prepared as well-defined, homogeneous materials of high purity, with minimal molecular weight dispersity (Zalipsky, S. et al., Bioconjugate Chem. 8:111 (1997); Wong, J. et al., Science 275:820 (1997)). The lipopolymer can also be a “neutral” lipopolymer, such as a polymer-distearoyl conjugate, as described in U.S. Pat. No. 6,586,001, incorporated by reference herein.

When a lipid-polymer conjugate is included in the liposomes, typically between 1-20 mole percent of the lipid-polymer conjugate is incorporated into the total lipid mixture (see, e.g., U.S. Pat. No. 5,013,556).

The liposomes can additionally include a lipopolymer modified to include a ligand, forming a lipid-polymer-ligand conjugate, also referred to herein as a ‘lipopolymer-ligand conjugate’. The ligand can be a therapeutic molecule, such as a drug or a biological molecule having activity in vivo, a diagnostic molecule, such as a contrast agent or a biological molecule, or a targeting molecule having binding affinity for a binding partner, preferably a binding partner on the surface of a cell. A preferred ligand has binding affinity for the surface of a cell and facilitates entry of the liposome into the cytoplasm of a cell via internalization. A ligand present in liposomes that include such a lipopolymer-ligand is oriented outwardly from the liposome surface, and therefore available for interaction with its cognate receptor.

Methods for attaching ligands to lipopolymers are known, where the polymer can be functionalized for subsequent reaction with a selected ligand. (U.S. Pat. No. 6,180,134; Zalipsky, S. et al., FEBS Lett. 353:71 (1994); Zalipsky, S. et al., Bioconjugate Chem. 4:296 (1993); Zalipsky, S. et al., J. Control. Rel. 39:153 (1996); Zalipsky, S. et al., Bioconjugate Chem. 8(2):111 (1997); Zalipsky, S. et al., Meth. Enzymol. 387:50 (2004)). Functionalized polymer-lipid conjugates can also be obtained commercially, such as end-functionalized PEG-lipid conjugates (Avanti Polar Lipids, Inc.). The linkage between the ligand and the polymer can be a stable covalent linkage or a releasable linkage that is cleaved in response to a stimulus, such as a change in pH or presence of a reducing agent.

The ligand can be a molecule that has binding affinity for a cell receptor or for a pathogen circulating in the blood. The ligand can also be a therapeutic or diagnostic molecule, in particular molecules that when administered in free form have a short blood circulation lifetime. In one embodiment, the ligand is a biological ligand, and preferably is one having binding affinity for a cell receptor. Exemplary biological ligands are molecules having binding affinity to receptors for CD4, folate, insulin, LDL, vitamins, transferrin, asialoglycoprotein, selectins, such as E, L, and P selectins, Flk-1,2, FGF, EGF, integrins, in particular, α₄β₁ αᵥβ₃, αᵥβ₁ αᵥβ₅, αᵥβ₆ integrins, HER2, and others. Preferred ligands include proteins and peptides, including antibodies and antibody fragments, such as F(ab′)₂, F(ab)₂, Fab′, Fab, Fv (fragments consisting of the variable regions of the heavy and light chains), and scFv (recombinant single chain polypeptide molecules in which light and heavy variable regions are connected by a peptide linker), and the like. The ligand can also be a small molecule peptidomimetic. It will be appreciated that a cell surface receptor, or fragment thereof, can serve as the ligand. Other exemplary targeting ligands include, but are not limited to vitamin molecules (e.g., biotin, folate, cyanocobalamine), oligopeptides, oligosaccharides. Other exemplary ligands are presented in U.S. Pat. Nos. 6,214,388, 6,316,024, 6,056,973, and 6,043,094, which are herein incorporated by reference.

B. Preparation of Liposome Formulation

A peptide boronic acid compound is accumulated and trapped inside the liposomes by formation of a boronate ester between hydroxyl functionalities on a liposome-entrapped compound of Formula I and the boronic acid compound (Eggert, H. et al., J. Org. Chem. 64:3846-52 (1999)). In brief, a compound of Formula I containing multiple hydroxyl functionalities is disposed inside the liposomes, the peptide boronic acid compound is diffused across the liposome lipid bilayer membrane, and a boronate ester is formed, entrapping the peptide boronic acid compound in the liposome.

In one embodiment, the process is driven by pH, where a lower pH (e.g. pH 6-7) outside the liposome and somewhat higher pH (pH 7.5-8.5) on the interior of the liposome, combined with the presence of a compound of Formula I or I, induces accumulation and loading of the compound. In this embodiment, the composition is prepared by formulating liposomes having a higher-inside/lower-outside gradient of a compound of Formula I or II. An aqueous solution of the compound of Formula I or II, selected as described above, is prepared at a desired concentration, determined as described above. It is preferred that the compound of Formula I or II when in solution have a viscosity suitable for lipid hydration, described below. The pH of the aqueous solution compound of Formula I or II is preferably greater than about 7.0.

The aqueous compound of Formula I or II solution is used for hydration of a dried lipid film, prepared from the desired mixture of vesicle-forming lipids, non-vesicle-forming lipids (such as cholesterol, DOPE, etc.), lipopolymer, such as mPEG-DSPE, and any other desired lipid bilayer components. A dried lipid film is prepared by dissolving the selected lipids in a suitable solvent, typically a volatile organic solvent, and evaporating the solvent to leave a dried film. The lipid film is hydrated with a solution containing the compound of Formula I or II, adjusted to a pH of greater than about 7.0, to form liposomes.

Examples 1-5 describe preparations of liposomes composed of the lipids egg phosphatidycholine (PC), cholesterol (CHOL) and polyethylene glycol derivatized distearolphosphatidyl ethanolamine (PEG-DSPE). The lipids, at a molar ratio of 10:5:1 PC:CHOL:PEG-DSPE are dissolved in chloroform and the solvent is evaporated to form a lipid film. The lipid film is hydrated with an aqueous solution of polyvinyl alcohol, pH 7.5, to form liposomes having the compound of Formula I or II entrapped inside.

After liposome formation, the liposomes can be sized to obtain a population of liposomes having a substantially homogeneous size range, typically between about 0.01 to 0.5 microns, more preferably between 0.03-0.40 microns. One effective sizing method for REVs and MLVs involves extruding an aqueous suspension of the liposomes through a series of polycarbonate membranes having a selected uniform pore size in the range of 0.03 to 0.2 micron, typically 0.05, 0.08, 0.1, or 0.2 microns. The pore size of the membrane corresponds roughly to the largest sizes of liposomes produced by extrusion through that membrane, particularly where the preparation is extruded two or more times through the same membrane. Homogenization methods are also useful for down-sizing liposomes to sizes of 100 nm or less (Martin, F. J., in Specialized Drug Delivery Systems—Manufacturing and Production Technology, P. Tyle, Ed., Marcel Dekker, New York, pp. 267-316 (1990)).

After sizing, unencapsulated bulk phase compound of Formula I or II is removed by a suitable technique, such as dialysis, centrifugation, size exclusion chromatography or ion exchange to achieve a suspension of liposomes having a high concentration of compound of Formula I or II inside and preferably little to no compound of Formula I or II outside. Also after liposome formation, the external phase of the liposomes is adjusted, by titration, dialysis or the like, to a pH of less than about 7.0.

The peptide boronic acid compound to be entrapped is then added to the liposome dispersion for active loading into the liposomes. The amount of peptide boronic acid compound added may be determined from the total amount of drug to be encapsulated, assuming 100% encapsulation efficiency, i.e., where all of the added compound is eventually loaded into liposomes in the form of boronate ester.

The mixture of the compound and liposome dispersion are incubated under conditions that allow uptake of the compound by the liposomes to a compound concentration that is several times that of the compound in the bulk medium, as evidence by the formation of precipitate in the liposomes. The latter may be confirmed, for example, by standard electron microscopy or X-ray diffraction techniques. Typically, the incubating is carried out at an elevated temperature, and preferably above the phase transition temperature Tₚ of the liposome lipids. For high-phase transition lipids having a Tₚ of 50° C., for example, incubation may be carried out at between 55-60° C. The incubation time may vary from between an hour or less to up to 12 hours or more, depending on incubation temperature.

At the end of this incubation step, the suspension may be further treated to remove free (non-encapsulated) compound, e.g., using any of the methods mentioned above for removing free polymer from the initial liposome dispersion containing entrapped compound of Formula I or II.

Examples 1-5 describe a method of preparing liposomes comprising a boronic acid compound and a compound of Formula I or II in the form of a boronate ester, where the compound of Formula I or II is a complexing agent. In these examples, a thin lipid film of egg PC and cholesterol is prepared. The lipid film is hydrated with a solution of a compound of Formula I or II to form liposomes having a compound of Formula I or II entrapped in the internal aqueous compartment. Unentrapped Formula I or II compound is removed by a suitable technique, such as dialysis, centrifugation, size exclusion chromatography or ion exchange to achieve a suspension of liposomes having a high concentration of the Formula I or II compound inside and preferably little to no Formula I or II compound outside. Then, the desired peptide boronic acid compound is added to the external medium. The compound in its unionized state is freely permeable across the liposomal lipid bilayers. Once inside the liposomes, the compound reacts with the entrapped Formula I or II compound to form a boronate ester, shifting the equilibrium toward passage of more drug across the lipid bilayer. In this way, the peptide boronic acid compound accumulates in the liposomes and in stably entrapped therein.

Liposome formulations that include a lipid-polymer-ligand targeting conjugate can be prepared by various approaches. One approach involves preparation of lipid vesicles that include an end-functionalized lipid-polymer derivative; that is, a lipid-polymer conjugate where the free polymer end is reactive or “activated” (see, for example, U.S. Pat. Nos. 6,326,353 and 6,132,763). Such an activated conjugate is included in the liposome composition and the activated polymer ends are reacted with a targeting ligand after liposome formation. In another approach, the lipid-polymer-ligand conjugate is included in the lipid composition at the time of liposome formation (see, e.g., U.S. Pat. Nos. 6,224,903 and 5,620,689). In yet another approach, a micellar solution of the lipid-polymer-ligand conjugate is incubated with a suspension of liposomes and the lipid-polymer-ligand conjugate is inserted into the pre-formed liposomes (see, e.g., U.S. Pat. Nos. 6,056,973 and 6,316,024).

### III. Methods of Use

The liposome formulation having a peptide boronic acid compound entrapped in the form of a boronate ester are used for treatment of tumor-bearing patients. In embodiments where the peptide boronic acid compound includes an isotope of boron, the liposome formulation can be used for boron neutron capture therapy. These uses will now be described.

A. Tumor Treatment

Boronic acid compounds are in the class of drugs referred to as proteasome inhibitors. Proteasome inhibitors induce apoptosis of cells by their ability to inhibit cellular proteasome activity. More specifically, in eukaryotic cells, the ubiquitin-proteasome pathway is the central pathway for protein degradation of intracellular proteins. Proteins are initially targeted for proteolysis by the attachment of a polyubiquitin chain, and then rapidly degraded to small peptides by the proteasome and the ubiquitin is released and recycled. This co-ordinated proteolytic pathway is dependent upon the synergistic activity of the ubiquitin-conjugating system and the 26S proteasome. The 26S proteasome is a large (1,500-2,000 kDa) multi-subunit complex present in the nucleus and cytoplasm of eukaryotes. The catalytic core of this complex, referred to as the 20S proteasome, is a cylindrical structure consisting of four heptameric rings containing α- and β-subunits. The proteasome is a threonine protease, the N-terminal threonine of the β-subunit providing the nucleophile that attacks the carbonyl group of the peptide bond in target proteins. At least three distinct proteolytic activities are associated with the proteasome: chymotryptic, tryptic and peptidylglutamyl. The ability to recognize and bind polyubiquitinated substrates is conferred by 19S (PA700) subunits, which bind to each end of the 20S proteasome. These accessory subunits unfold substrates and feed them into the 20S catalytic complex, whilst removing the attached ubiquitin molecules. Both the assembly of the 26S proteasome and the degradation of protein substrates are ATP-dependent (Almond, Leukemia 16:433 (2002)).

The ubiquitin-proteasome system regulates many cellular processes by the coordinated and temporal degradation of proteins. By controlling levels of many key cellular proteins, the proteasome acts as a regulator of cell growth and apoptosis and disruption of its activity has profound effects on the cell cycle. For example, defective apoptosis is involved in the pathogenesis of several diseases including certain cancers, such as B cell chronic lymphocytic leukemia, where there is an accumulation of quiescent tumor cells.

Proteasome inhibitors as a class of compounds in general act by inhibiting protein degradation by the proteasome. The class includes peptide aldehydes, peptide vinyl sulfones, which act by binding to and directly inhibiting active sites within the 20S core of the proteasome. Peptide aldehydes and peptide vinyl sulfones, however, bind to the 20S core particle in an irreversible manner, such that proteolytic activity cannot be restored upon their removal. In contrast, peptide boronic acid compounds confers stable inhibition of the proteasome, yet dissociates slowly from the proteasome. The peptide boronic acid compounds are more potent than their peptide aldehyde analogs, and act more specifically in that the weak interaction between boron and sulfur means that peptide boronates do not inhibit thiol proteases (Richardson, P. G., et al., Cancer Control. 10(5):361, (2003)).

Exposure of a variety of tumor-derived cell lines to proteasome inhibitors triggers apoptosis, likely as a result of effects on several pathways, including cell cycle regulatory proteins, p53, and nuclear factor kappa B (NF-κB) (Grimm, L. M. and Osborne, B. A., Results Probl. Cell. Differ. 23:209-28 (1999); Orlowski, R. Z., Cell Death Differ. 6(4):303-13 (1999)). Many of the initial studies documenting proteasome inhibitor-mediated apoptosis used cells of hematopoietic origin, including monoblasts (Imajoh-Ohmi, S. et al., Biochem. Biophys. Res. Commun. 217(3):1070-77 (1995)), T-cell and lymphocytic leukemia cells (Shinohara, K. et al., Biochem. J. 317(Pt 2):385-88, (1996)), lymphoma cells (Tanimoto, Y. et al., J. Biochem. (Tokyo) 121 (3):542-49 (1997)), and promyelocytic leukemia cells (Drexler, H. C., Proc. Natl. Acad. Sci. U.S.A. 94(3):855-60 (1997)). The first demonstration of in vivo antitumor activity of a proteasome inhibitor used a human lymphoma xenograft model (Orlowski, R. Z. et al., Cancer Res 58(19):4342-48 (1998)). Furthermore, proteasome inhibitors were reported to induce preferential apoptosis of patient-derived lymphoma (Orlowski, R. et al. Cancer. Res., 58:(19):4342 (1998)) and leukemia cells (Masdehors, P. et al., Br. J. Haematol. 105(3):752-57 (1999)) and to preferentially inhibit proliferation of multiple myeloma cells (Hideshima, T. et al., Cancer Res., 61(7): 3071-76 (2001)) with relative sparing of control, non-transformed cells. Thus, proteasome inhibitors are particularly useful as therapeutic agents in patients with refractory hematologic malignancies.

In one embodiment, a liposome formulation comprising a peptide boronic acid compound is used for treatment of cancer, and more particularly for treatment of a tumor in a cancer patient.

Multiple myeloma is an incurable malignancy that is diagnosed in approximately 15,000 people in the United States each year (Richardson, P. G. et al., Cancer Control. 10(5):361 (2003)). It is a hematologic malignancy typically characterized by the accumulation of clonal plasma cells at multiple sites in the bone marrow. The majority of patients respond to initial treatment with chemotherapy and radiation, however most eventually relapse due to the proliferation of resistant tumor cells. In one embodiment, the invention provides a method for treating multiple myeloma by administering a liposome formulation comprising a peptide boronic acid compound entrapped in the form a boronate ester.

The liposome formulation is also effective in breast cancer treatment by helping to overcome some of the major pathways by which cancer cells resist the action of chemotherapy. For example, signaling through NF-kB, a regulator of apoptosis, and the p44/42 mitogen-activated protein kinase pathway, can be anti-apoptotic. Since proteasome inhibitors block these pathways, the compounds are able to activate apoptosis. Thus, the invention provides a method for treating a subject having breast cancer, by administering liposomes comprising a peptide boronic acid compound. Moreover, since chemotherapeutic agents such as taxanes and anthracyclines have been shown to activate one or both of these pathways, use of a proteasome inhibitor in combination with conventional chemotherapeutic agents acts to enhance the antitumor activity of drugs, such as paclitaxel and doxorubicin. Thus, in another embodiment, the invention provides a treatment method where a chemotherapeutic agent, in free form or in liposome-entrapped form, is administered in combination with a liposome-entrapped peptide boronic acid compound.

Doses and a dosing regimen for the liposome formulation will depend on the cancer being treated, the stage of the cancer, the size and health of the patient, and other factors readily apparent to an attending medical caregiver. Moreover, clinical studies with the proteosome inhibitor bortezomib, Pyz-Phe-boroLeu (PS-341), provide ample guidance for suitable dosages and dosing regimens. For example, given intravenously once or twice weekly, the maximum tolerated dose in patients with solid tumors was 1.3 mg/m² (Orlowski, R. Z. et al., Breast Cancer Res. 5:1-7 (2003)). In another study, bortezomib given as an intravenous bolus on days 1, 4, 8, and 11 of a 3-week cycle suggested a maximum tolerated dose of 1.56 mg/m² (Vorhees, P. M. et al., Clinical Cancer Res. 9:6316 (2003)).

The liposome formulation is typically administered parenterally, with intravenous administration preferred. It will be appreciated that the formulation can include any necessary or desirable pharmaceutical excipients to facilitate delivery.

In the treatment methods described above, a preferred proteosome inhibitor is bortezomib, Pyz-Phe-boroLeu; Pyz: 2,5-pyrazinecarboxylic acid; PS-341), having the structure:

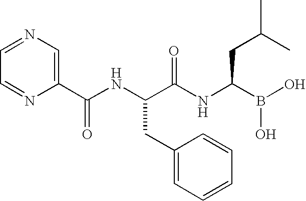

Bortezomib has been shown to have activity against a variety of cancer tissues, including breast, ovarian, prostate, lung, and against various tumors, such as pancreatic tumors, lymphomas and melanoma. (Teicher, B. A. et al., Clin. Cancer Res. 5(9):2638-45 (1999); Adams, J., Semin. Oncol., 28(6):613-19 (2001); Orlowski, R. Z. and Dees, E. C., Breast Cancer Res. 5(1):1-7 (2002); Frankel et al., Clin. Cancer Res. 6(9):3719-28 (2000); Shah, S. A. et al., J. Cell. Biochem. 82(1):110-22 (2001)).

B. Boron Neutron Capture Therapy

In another aspect, a method of administering a boron-10 isotope to a tumor, for boron-neutron capture therapy (¹⁰B-NCT), is provided. Neutron-capture therapy for cancer treatment is based on the interaction of ¹⁰B isotope with thermal neutron, each relatively innocuous, according to the following equation:

¹⁰B+¹n→⁷Li+⁴He+2.4MeV

The reaction results in intense ionizing radiation that is confined to single or adjacent cancer cells. Thus, for successful treatment, it is desirable to deliver adequate amounts of a boron-10 isotope to tumors. The liposome formulation described herein provides a means to entrap a peptide boronic acid compound bearing a ¹⁰B isotope in a liposome. Liposomes that include a surface coating a hydrophilic polymer chains accumulate preferentially in tumors, due to the long blood circulation lifetime of such liposomes (see, e.g., U.S. Pat. Nos. 5,013,556 and 5,213,804). The liposomes loaded with a peptide boronic acid compound bearing a ¹⁰B isotope eradicate tumors by two independent mechanisms: the liposomes act as a drug reservoir in the tumor and gradually liberate the anti-cancer compound in the tumor and the liposomes serve to accumulate sizable amounts of boron-10 isotope in the tumor assisting the efficacy of boron neutron capture therapy.

From the foregoing, the various aspects and features of the invention are apparent. Liposomes comprising a water-soluble, lipid bilayer impermeable compound of Formula I or II and a peptide boronic acid compound are described. The liposomes are prepared by encapsulating the compound of Formula I or II in the internal aqueous compartments of liposomes, removing any unencapsulated compound of Formula I or II from the external medium, adding the lipid bilayer permeable boronic acid compound, which passes through the lipid bilayer membrane to form a reversible ester bond with the vicinal hydroxyl moieties on the compound of Formula I or II. In this way, boronic acid compound, which is normally freely permeable across the lipid bilayer, is stably entrapped in the liposomes. Accumulation of the peptide boronic acid compound into the liposomes occurs in the absence of an ion gradient, however, an ion gradient can be present if desired.

### EXAMPLES

The following examples further illustrate the invention described herein and are in no way intended to limit the scope of the invention.

### Example 1

### Synthesis of Compound 1 Complexing Reagent

Lactose (4.1 g, 12 mmol), methylamine hydrochloride (1.35 g, 20 mmol), and sodium cyanoborohydride (5 M, 1.2 mL, 6 mmol) were added into a pressure tube, filled by Argon, adjusted pH to 7.0 (pH paper). The reaction tube was sealed, and the reaction mixture was stirred at room temperature for 24 h, and then heated at 40-50° C. for 16 h. The reaction was monitored by TLC (SiO2, ethanol/acetic acid/water 5:1:2). The reaction mixture was precipitated in ethanol (400 mL). The precipitate was separated by filtration, and purified on an ion-exchange resin column (Bio-Rex 70, water used as eluant). The product was purified twice by ion-exchange. Product fractions were combined, and lyophilized to give 1.373 g of the product as a white solid. ¹H NMR (400 MHz, D₂O) δ 4.53 (d, J=8 Hz, 1H), 4.23-4.19 (m, 1H), 3.97-3.53 (m, 10H), 3.55 (dd, J=10 and 8 Hz, 1H), 3.361 (dd, J=13 and 3 Hz, 1H), 3.14 (dd, J=13 and 10 Hz, 1H), 2.77 (s, 3H).

### Liposomes Loaded with Bortezomib

Compound 1 was dissolved in water and adjusted to pH 7.4. A mixture of egg phosphatidyl choline, cholesterol, and polyethylene glycol-distearoylphosphatidylethanolamine (PEG-DSPE, PEG molecular weight 2,000 Da, Avanti Polar Lipids, Birmingham, Ala.) in a molar ratio of 10:5:1 was dissolved in chloroform, the solvent was evaporated in vacuum, the lipid film was incubated with shaking in the Compound 1 solution, and the lipid dispersion was extruded under pressure through 2 stacked Nucleopore (Pleasanton, Calif.) membranes with pore size 0.2 μm. The outer buffer was exchanged for NaCl 0.14 M containing 5 mM of sodium hydroxyethylpiperazine-ethane sulfonate (HEPES) at pH 6.5 using gel chromatography on Sepharose CL-4B (Pharmacia, Piscataway, N.J.); at the same time, unentrapped Compound 1 was removed. To the so obtained liposomes, bortezomib was added. The mixture was incubated overnight at 37° C. with shaking, treated with Dowex 50W×4 (Sigma Chemical Co., St. Louis, Mo.), and equilibrated with NaCl-HEPES solution to remove non-encapsulated bortozemib. The resulting liposomes were sterilized by filtration through a 0.2 μm filter.

### Example 2

### Synthesis of Compound 2 Complexing Reagent

Lactose (4.79 g, 14 mmol), L-lysine (0.985 g, 6 mmol), sodium cyanoborohydride (5 M, 3 mL, 15 mmol), and MilliQ water (8 mL) were added to a pressure tube containing a magnetic stirring bar. The tube was filled by Argon, sealed, and stirred at 50-60° C. for 2 days. The reaction mixture was precipitated in ethanol. The precipitate was separated, and then dissolved in MilliQ water, added to a Dialysis tube (MWCO=500), dialyzed against water. The product was precipitated in ethanol again, filtered, and the solid was dried in vacuum for 2 days to afford 3.081 g of the product as a white solid. ¹H NMR (400 MHz, D₂O) δ 4.52 (d, J=6 Hz, 1H), 4.50 (d, J=6 Hz, 1H), 4.25-4.12 (m, 2H), 3.97-3.50 (m, 28H), 3.07 (m, 2H), 1.70 (broad. m, 4H), 1.40 (broad m, 2H).

### Liposomes Loaded with Bortezomib

Compound 2 was dissolved in water and adjusted to pH 7.4. A mixture of egg phosphatidyl choline, cholesterol, and polyethylene glycol-distearoylphosphatidylethanolamine (PEG-DSPE, PEG molecular weight 2,000 Da, Avanti Polar Lipids, Birmingham, Ala.) in a molar ratio of 10:5:1 was dissolved in chloroform, the solvent was evaporated in vacuum, the lipid film was incubated with shaking in the Compound 2 solution, and the lipid dispersion was extruded under pressure through two stacked Nucleopore (Pleasanton, Calif.) membranes with pore size 0.2 μm. The outer buffer was exchanged for NaCl 0.14 M containing 5 mM of sodium hydroxyethylpiperazine-ethane sulfonate (HEPES) at pH 6.5 using gel chromatography on Sepharose CL-4B (Pharmacia, Piscataway, N.J.); at the same time, unentrapped Compound 2 was removed. To the so obtained liposomes, bortezomib was added. The mixture was incubated overnight at 37° C. with shaking, treated with Dowex 50W×4 (Sigma Chemical Co., St. Louis, Mo.), and equilibrated with NaCl-HEPES solution to remove non-encapsulated bortozemib. The resulting liposomes were sterilized by filtration through a 0.2 μm filter.

### Example 3

### Synthesis of Compound 3 Complexing Reagent

Dextrose (4.5 g, 25 mmol), L-lysine (0.985 g, 6 mmol), sodium cyanoborohydride (5 M, 5 mL, 25 mmol), and MilliQ water (8 mL) were added to a pressure tube containing a magnetic stirring bar. The tube was filled by Argon, sealed, and stirred at 50-60° C. for 2 days. The reaction mixture was precipitated in ethanol. The precipitate was separated, and then dissolved in MilliQ water, added to a Dialysis tube (MWCO=500), dialyzed against water. The product was precipitated in ethanol again, filtered, and the solid was dried in vacuum for 16 h to afford 3.22 g of the product as a white solid. ¹H NMR (400 MHz, D₂O) δ 3.84-3.60 (m, 15H), 3.09 (broad m, 4H), 1.70 (broad. m, 4H), 1.40 (broad m, 2H).

### Liposomes Loaded with Bortezomib

Compound 3 was dissolved in water and adjusted to pH 7. A mixture of egg phosphatidyl choline, cholesterol, and polyethylene glycol-distearoylphosphatidylethanolamine (PEG-DSPE, PEG molecular weight 2,000 Da, Avanti Polar Lipids, Birmingham, Ala.) in a molar ratio of 10:5:1 was dissolved in chloroform, the solvent was evaporated in vacuum, the lipid film was incubated with shaking in the Compound 3 solution, and the lipid dispersion was extruded under pressure through 2 stacked Nucleopore (Pleasanton, Calif.) membranes with pore size 0.2 μm. The outer buffer was exchanged for NaCl 0.14 M containing 5 mM of sodium hydroxyethylpiperazine-ethane sulfonate (HEPES) at pH 6.5 using gel chromatography on Sepharose CL-4B (Pharmacia, Piscataway, N.J.); at the same time, unentrapped Compound 3 was removed. To the so obtained liposomes, bortezomib was added. The mixture was incubated overnight at 37° C. with shaking, treated with Dowex 50W×4 (Sigma Chemical Co., St. Louis, Mo.), and equilibrated with NaCl-HEPES solution to remove non-encapsulated bortozemib. The resulting liposomes were sterilized by filtration through a 0.2 μm filter.

### Example 4

### Synthesis of Compounds 4 Complexing Reagent

Dendrimer (PAMAM, second generation, M.W. 3284, 20% wt. in MeOH, 2 g) was evaporated under reduced pressure. The residue was dissolved in water (5 mL), transferred to a pressure tube, and adjusted pH to 7.0 with hydrochloric acid. Dextrose (0.51 g, 2.83 mmol) and sodium cyanoborohydride (5 M, 3 mL, 15 mmol) were added to a pressure tube containing a magnetic stirring bar. The tube was filled with argon, sealed, and stirred at 40° C. for 16 hours. The reaction mixture was precipitated in ethanol. The precipitate was separated, and then dissolved in MilliQ water, added to a dialysis tube (MWCO=1,000), dialyzed against water. Then the product was precipitated in ethanol, filtered, and the solid was dried in vacuum for 16 hours to afford 0.48 g of the product as a white solid. The dendrimer used had 16 amino groups per molecule. M.W. 3284. Compound 4 was a mixture of several different numbers of conjugates.

### Liposomes Loaded with Bortezomib

Compound 4 was dissolved in water and adjusted to pH 7.4. A mixture of egg phosphatidyl choline, cholesterol, and polyethylene glycol-distearoylphosphatidylethanolamine (PEG-DSPE, PEG molecular weight 2,000 Da, Avanti Polar Lipids, Birmingham, Ala.) in a molar ratio of 10:5:1 was dissolved in chloroform, the solvent was evaporated in vacuum, the lipid film was incubated with shaking in the Compound 4 solution, and the lipid dispersion was extruded under pressure through 2 stacked Nucleopore (Pleasanton, Calif.) membranes with pore size 0.2 μm. The outer buffer was exchanged for NaCl 0.14 M containing 5 mM of sodium hydroxyethylpiperazine-ethane sulfonate (HEPES) at pH 6.5 using gel chromatography on Sepharose CL-4B (Pharmacia, Piscataway, N.J.); at the same time, unentrapped Compound 4 was removed. To the so obtained liposomes, bortezomib was added. The mixture was incubated overnight at 37° C. with shaking, treated with Dowex 50W×4 (Sigma Chemical Co., St. Louis, Mo.), and equilibrated with NaCl-HEPES solution to remove non-encapsulated bortozemib. The resulting liposomes were sterilized by filtration through a 0.2 μm filter.

### Example 5

### Synthesis of Compound 5 Complexing Reagent

Lactose (5.13 g, 25 mmol), 3-amino-1,2-propanediol (1.64 g, 18 mmol), sodium cyanoborohydride (5 M, 3.6 mL, 18 mmol), and MilliQ water (6 mL) were added to a pressure tube containing a magnetic stirring bar. The tube was filled by Argon, sealed, and stirred at 40-50° C. for 2 days. The reaction mixture was precipitated in methanol. The precipitate was separated, and then dissolved in MilliQ water, added to a Dialysis tube (MWCO=100), dialyzed against water. Then the product was precipitated in ethanol, filtered, and the solid was dried in vacuum for 16 h to afford 1.07 g of the product as a white solid. ¹H NMR (400 MHz, D₂O) δ 3.84-3.60 (m, 15H), 3.09 (broad m, 4H), 1.70 (broad. m, 4H), 1.40 (broad m, 2H).

### Liposomes Loaded with Bortezomib

Compound 5 was dissolved in water and adjusted to pH 7.4. A mixture of egg phosphatidyl choline, cholesterol, and polyethylene glycol-distearoylphosphatidylethanolamine (PEG-DSPE, PEG molecular weight 2,000 Da, Avanti Polar Lipids, Birmingham, Ala.) in a molar ratio of 10:5:1 was dissolved in chloroform, the solvent was evaporated in vacuum, the lipid film was incubated with shaking in the Compound 5 solution, and the lipid dispersion was extruded under pressure through 2 stacked Nucleopore (Pleasanton, Calif.) membranes with pore size 0.2 μm. The outer buffer was exchanged for NaCl 0.14 M containing 5 mM of sodium hydroxyethylpiperazine-ethane sulfonate (HEPES) at pH 6.5 using gel chromatography on Sepharose CL-4B (Pharmacia, Piscataway, N.J.); at the same time, unentrapped Compound 5 was removed. To the so obtained liposomes, bortezomib was added. The mixture was incubated overnight at 37° C. with shaking, treated with Dowex 50W×4 (Sigma Chemical Co., St. Louis, Mo.), and equilibrated with NaCl-HEPES solution to remove non-encapsulated bortozemib. The resulting liposomes were sterilized by filtration through a 0.2 μm filter.

### Example 6

### In Vitro Activity of Liposome-Entrapped Bortezomib

Multiple myeloma cells are grown to confluence on microtiter plates. The cells are incubated with liposomes prepared as described in Examples 1-5 at various concentrations of peptide boronic acid compound. After a 24 hour incubation period, the cells are inspected for apoptosis. It is found that cells treated with the liposome formulation have a higher incidence of apoptosis than control cells.

### Example 7

### In Vitro Activity of Liposome-Entrapped Bortezomib

Several in vitro studies were performed on the liposomes prepared as described in Examples 1-5 using a liposome-entrapped bortezomib with meglumine as a complexing reagent as a comparison. Data was generated from (1) ITC binding studies of the bortezomib to the complexing reagents employed in the liposomes, (2) drug release studies from the liposomes; and (3) drug loading stability studies.

### ITC Drug Binding Study

The binding of the various complexing reagents to bortazomib were investigated by isothermal titration calorimetry (VP-ITC, MicroCal, Northampton, Mass.) under controlled parameters. The complexing reagents from Examples 1-5 meglumine (1 mM) and bortezomib (18.2 mM), solutions were individually prepared dissolving the desired amount of materials in the same glycine stock (Sigma-Aldrich, 100 mM, pH 9.5) to ensure matching of the glycine concentration and the pH. The pH level was checked twice and adjustments were made as necessary to within 0.2 units. The solutions were degassed for 10-15 min. The bortezomib solution was loaded into the automated syringe and titrated (5-10 μL per injection) to the complexing reagent (CR) solution that was loaded in the ITC sample cell. The experiments were conducted at 30° C. The reference run was performed with titration of the same drug solution into the 100 mM glycine solution using the same run parameters used for the active runs. Data processing to generate the binding parameters was performed using the software program provided by the ITC manufacture (Microcal origin v5.0).

| | | K | ΔH |
|---|---|---|---|
| CR | pH | (mol⁻¹) | (Kcal/mol) |
| Meglumine | 9.5 | 5526 | −4.1 |
| Example 2 | 9.5 | 2589 | −6.9 |
| Example 3 | 9.5 | 1350 | −9.1 |
| Example 4 | 9.5 | 1569 | −5.4 |
| Example 5 | 9.5 | 2366 | −6.3 |
| Example 1 | 9.5 | 10810 | −9.4 |

### In Vitro Drug Release Assay in Whole Blood

A mixture of a test formulation and rat whole blood in a volume ratio of 1:4 was rocked at 500 rpm, at 37° C. for 24 hrs. Samples were pulled out at time points 0, 1, 2, 6, and 24 hrs, and spun at low RPM for several minutes. The supernatant plasma was taken and the free drug was analyzed with MS/MS. There was no significant difference noted from the drug released between liposomes prepared according to Example 1 and liposome-entrapped bortezomib with meglumine as a complexing reagent.

### Drug Loading Stability Study

The liposome encapsulated bortazomib formulations were incubated at 25° C., and checked with particle size, pH, and the drug encapsulation efficiency. The drug encapsulation efficiency was determined with a size exclusion chromatography. The sample (100 uL) was loaded in a Bio-Gel, P-6 column (0.5×30 cm), eluted with pH 7.0, 100 mM HEPES-NaCl (150 mM) solution, and collected fractions (1 mL/each). The liposomal and free drug fractions were checked by UV at 270 nm, and combined respectively, and then analyzed by HPLC.

Liposomal Drug Loading Stabulity Study at 25° C.

| | | E.E. | E.E. | E.E. | E.E. |
|---|---|---|---|---|---|
| Lot # | CR | (day 1) | (1 month) | (2 month) | (4 month) |
| 1 | Meglumine | 99% | 85% | 13% | n/a |
| 2a | Example 1 | 98% | 98% | n/a | 96% |
| 2b | Example 1 | 99% | 99% | n/a | 99% |

E.E. = Encapsulation Efficiency

Although the invention has been described with respect to particular embodiments, it will be apparent to those skilled in the art that various changes and modifications can be made without departing from the invention.

It is claimed:

1. A composition, comprising
liposomes formed of a vesicle-forming lipid, and
entrapped in said liposomes, a boronate ester compound comprised of a peptide boronic acid compound and a compound of Formula I or II.

2. The composition of claim 1, wherein said peptide boronic acid compound is a dipeptidyl boronic acid compound.

3. The composition of claim 1, wherein said peptide boronic acid compound is bortezomib.

4. The composition of claim 1, comprising a Formula II compound.

5. The composition of claim 4, wherein R₁, R₂, R₃, R₄ and R₅ are H.

6. The composition of claim 5, wherein D is a conjugated G₂ dendrimer which has 16 amino groups per molecule.

7. The composition of claim 1, comprising a Formula I compound.

8. The composition of claim 7, wherein Y is H.

9. The composition of claim 7, wherein Y is B.

10. The composition of claim 7, wherein Y is E.

11. The composition of claim 8, wherein R₁ is H.

12. The composition of claim 8, wherein R₂ is H.

13. The composition of claim 8, wherein R₃ is H.

14. The composition of claim 8, wherein R₃ is A.

15. The composition of claim 8, wherein R₄ is H.

16. The composition of claim 8, wherein R₅ is H.

17. The composition of claim 8, wherein Y, R₁, R₂, R₄, R₅ are each H and R₃ is A.

18. The composition of claim 1, wherein said liposomes further comprise a higher inside/lower outside ion gradient.

19. The composition of claim 18, wherein said ion gradient is a hydrogen ion gradient.

20. The composition of claim 19, wherein said hydrogen ion gradient provides an inside pH of between about 7.5-8.5 and an outside pH of between about 6-7.

21. The composition of claim 1, wherein said liposomes further comprise between about 1-20 mole percent of a hydrophobic moiety derivatized with a hydrophilic polymer.

22. The composition of claim 21, wherein said hydrophobic moiety derivatized with a hydrophilic polymer is a hydrophobic moiety derivatized with polyethylene glycol.

23. The composition of claim 22, wherein said hydrophobic moiety is a lipid.

24. A method of delivering a peptide boronic acid compound, comprising preparing a suspension of liposomes in an aqueous solution, said liposomes having an entrapped peptide boronic acid compound covalently attached to a compound of Formula I or II to form a peptidyl boronate ester compound,
administering the suspension of liposomes to a subject.

25. The method of claim 24, wherein said preparing further includes preparing a suspension of liposomes having a higher inside/lower outside ion gradient and adding said peptide boronic acid compound to the suspension to achieve uptake of the peptide boronic acid compound into the liposomes for formation of the peptidyl boronate ester compound.

26. The method of claim 24, wherein said preparing further includes preparing a suspension of liposomes having an external surface coating of hydrophilic polymer chains.

27. The method of claim 24, wherein said preparing further includes preparing a suspension of liposomes having an entrapped dipeptidyl boronic acid compound.

28. The method of claim 24, wherein said dipeptidyl boronic acid compound is bortezomib.

29. The method of claim 24, wherein said preparing further includes preparing a suspension of liposomes having an entrapped compound of Formula I wherein the compound is where Y, R₁, R₂, R₄, R₅ are H and R₃ is A.

30. The method of claim 24, wherein said administering is via injection.

31. A method of selectively destroying tumor tissue in a tumor-bearing subject undergoing radiation therapy, comprising
administering to a tumor-bearing subject, liposomes having an entrapped peptide boronic acid covalently attached to a compound of Formula I or II to form a peptidyl boronate ester compound and an isotope of boron; and
subjecting said subject to radiation therapy.

32. The method of claim 31, wherein said isotope of boron is in the peptide boronic acid.

33. The method of claim 31, wherein said isotope of boron is a ¹⁰B.
